(19) United States
(12) Patent Application Publication
(10) Pub. No.: US 2004/0220527 A1
(43) Pub. Date: Nov. 4, 2004

(54) Animal treatment and marking system

(76) Inventors: Paul Fleming Buckley, Auckland (NZ); Duncan Kekewich Jordan, Hamilton (NZ); Sahun Plant, Hamilton (NZ)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/478,968

(22) PCT Filed: May 28, 2002
(86) PCT No.: PCT/NZ02/00104

(30) Foreign Application Priority Data
May 29, 2001 (NZ) 512038
Sep. 21, 2001 (NZ) 514339

(51) Int. Cl. A61M 5/00

(52) U.S. Cl. 604/191

(57) ABSTRACT

A method of treating an animal with a single treatment device (1), including a primary (medical) treatment (8) and a secondary (marking) treatment (6), characterised in applying the secondary (marking) treatment with the device prior to applying the primary (medical) treatment with the same device.

### TECHNICAL FIELD

This invention relates to an improved animal marking system

In particular, this invention relates to an animal mark system for use with an animal treatment device such as a drench gun, or preferably a vaccinating syringe.

Reference throughout the specification shall be made to use of the present invention for marking an animal during treatment with a vaccinator. However, this should not be seen to be a limitation on the present invention in any way.

### BACKGROUND ART

Animal treatments such as vaccinations and drenching are an integral part of modern farming, ensuring the health and productivity of livestock operations.

Marking systems are often used during animal treatment to identify animals as having been treated to avoid situations such as some animals being left untreated, while over treating others.

Animal treatments, especially injections, preferably use a sterilising system to reduce the chances of cross contamination between animals as the same device is often used to treat a large number of animals.

Sterilising systems may involve swabbing the treatment area with a separate sterilising substance prior to treatment, or may involve the use of a sterilising system combined with the treatment device.

One such sterilising system is NZ 197866, which describes the use of a detachable cap incorporating a sterilising substance, which attaches onto the end of an injection device.

Prior to injection, the needle passes through an absorbent material impregnated with a sterilising substance before puncturing the skin of the animal.

This device was an improvement over existing sterilising systems, but also has some disadvantages.

The cap is designed to fit over the end of an injection gun, enabling the skin of the animal to also be swabbed with the sterilising substance prior to injection, in addition to swabbing the needle as it passes through. However, the outer surface of the cap quickly becomes contaminated by contact with the animal's skin and blood, which in turn contaminates the needle each time it passes through, requiring the cap to be frequently replaced.

A number of marking systems are known and may be separate from the treatment device and applied immediately afterwards to mark the site of treatment, such as with a chalk mark or paint stick.

However, this requires both hands of the farmer to be occupied, making it difficult to control the animal during handling, which may result in some animals being left unmarked and thus creating confusion as to which animals have been treated.

This could result in some animals being left untreated, or being treated more than once, both outcomes having a detrimental effect on the animal.

The above technique can also be time consuming which is a potential problem where the herd is large and is required to be treated within a narrow timeframe.

Other systems have been developed wherein the marking device is combined with the treatment device.

This enables the farmer to treat and mark the animals one-handed, which is a significant improvement as it allows the free hand to control the animal during treatment.

Various devices are known, such as New Zealand Patent No. 205823 wherein the animal is treated and immediately thereafter marked by an ink or aerosol cartridge located in the handle of the treatment device. However, animal treatments usually involve some level of discomfort to the animal, creating a situation where the animal may escape the control of the farmer after being treated and before being marked, or the mark being applied in a different location to the treatment.

Thus it has become preferable to use marking systems that mark the animal simultaneously with treatment.

A number of such devices are known. NZ Patent No. 266891 describes a device wherein an absorbent pad soaked in marking dye is fitted around the nozzle of a treatment device, such as an injector or drench gun. When the animal is treated, the pad is pressed against the animal leaving an identifying mark.

Other systems incorporate the use of ink syringes, such as U.S. Pat. No. 5,961,494, which involves a trigger handle that is squeezed, simultaneously actuating a treatment syringe and the ink syringe such that as the animal is treated, it is also marked in the approximate area of the treatment.

There are however a number of disadvantages associated with the aforementioned systems.

Systems that rely on physical contact require a certain amount of pressure to be applied to release the marking dye.

They also rely on the pad being able to release enough marker quickly to be visible.

There may also be an additional problem of having to reapply dye to the pad or syringe and have it in sufficient concentration to effect a clear mark These problems can often affect the ability of the device to mark the animal satisfactorily.

The use of these devices often allows the marking substance to enter the wound crated by the needle, which may potentially result in infection.

Devices that rely on the use of fluid markers also require a reservoir of marker to be carried that requires some form of pressure or propellant to drive the marker.

Such systems may also become clogged, require refilling and need effective pressurants to ensure the device operates consistently throughout the entire treatment period.

A large fluid reservoir may need to be carried on the back of the operator, hindering their movement.

Animal treatments often take place in stockyards where space is limited, thus bulky equipment can be a safety concern if animals become distressed, requiring the operator to quickly back away.

Ink syringe systems such as U.S. Pat. No. 5,961,494 also typically suffer several further problems.

After a number of uses the movement of the syringe piston becomes stiff and will eventually jam, requiring time consuming maintenance.

Another problem with syringe systems is that after a number of operations the piston action becomes stiff and will not return fully to its initial position, resulting in less than the metered dose of solutions/marker being delivered.

The marker itself is usually paint or ink based, and as such has no additional features other than acting as a visible indicator.

Accordingly, there are disadvantages even with systems attempting to simultaneously mark and treat the animal.

It is an object of the present invention to address the foregoing problems or at least to provide the public with a useful choice.

Further aspects and advantages of the present invention will become apparent from the ensuing description which is given by way of example only.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

### DISCLOSURE OF INVENTION

According to one aspect of the present invention there is provided a method of treating an animal with a single animal treatment device, including

a primary treatment, and

a secondary treatment

characterised by the step of

applying the secondary treatment with the device prior to applying the primary treatment with the same device.

According to another aspect of the present invention there is provided a method for applying the secondary treatment,

characterised by the further steps of:

a) pressing the animal treatment device against an animal,

b) retracting a needle shroud,

wherein the action of retracting the shroud activates an actuator mechanism which in turn depresses a reservoir nozzle for a determined length of time to apply a quantity of the secondary treatment

The primary treatment may be any treatment given to an animal, but will preferably be applied by vaccination.

The term “animal treatment device” may be any device capable of applying an animal treatment but will preferably be a vaccinating syringe, hereinafter referred to as a vaccinator.

However, it should be appreciated that the treatment device may be a device suitable for use in animal treatments, such as a drench gun, bolus applicator or so forth.

The secondary treatment will preferably be applied topically. However, this should not be seen as a limitation on the present invention in any way.

In preferred embodiments the secondary treatment will be used as a visible identification to mark an animal receiving the primary treatment.

However, this should not be seen as a limitation on the present invention in any way for the secondary treatment may alternatively have at least one of anti-microbial activity, cleansing properties, and/or anaesthetic properties, in conjunction with a visible identification or without.

As such, the definition of the terms “mark” and “marking system” should also not been seen as being limited to a visible identification, but rather the topical application of a treatment to an animal substantially as described above.

The secondary treatment may be applied in a variety of forms dependent on the nature of the applicator, including but not limited to colloidal suspensions, liquids or aerosols.

According to another aspect of the present invention there is provided an animal treatment device substantially as described above including,

an actuator,

a primary treatment applicator,

a secondary treatment applicator,

characterised in that

the treatment device is configured such that as the primary treatment is initiated, the actuator mechanism activates the application of the secondary treatment prior to the application of the primary treatment.

In preferred embodiments of the present invention the secondary treatment applicator includes a needle shroud that moves longitudinally with respect to and which protrudes beyond the needle of the vaccinator.

The needle shroud is preferably connected to a cap that attaches to the end of the vaccinator barrel. This allows the shroud to be pushed back as the vaccinator is pressed against an animal, activating the application of the secondary treatment before the vaccinator needle is inserted into the animal.

As the needle is withdrawn from the animal, a biasing means will preferably push the shroud back over the needle, protecting the operator from needle stick injuries, and the needle itself from damage or dirt if the vaccinator is dropped or placed on a surface.

Preferably the biasing material will be a spring, though in other embodiments the spring may be replaced by another material capable of forcing the shroud back over the needle, such as flexible plastic or spring steel.

In some embodiments the shroud is moulded with an external thread. This allows the shroud when depressed into the cap to rotate and push an actuator arm of the secondary treatment applicator and apply the secondary treatment.

Preferably, the thread length enables the secondary treatment applicator to be actuated for a determined duration to apply a metered dose of the secondary treatment. Upon removing the needle the shroud will rotate and return to its original position, relocating the actuator arm onto the thread in preparation for another treatment.

In other embodiments the needle shroud may have a projection which protrudes from the shroud at a substantially perpendicular angle. As the shroud is pushed back, the projection comes into contact with the actuator mechanism of the secondary treatment applicator.

The actuator mechanism preferably includes a lever that activates the application of the secondary treatment.

In these embodiments the actuator mechanism consists of a first actuator arm and a fulcrum at one end about which the arm can rotate. The first actuator arm may be of any material strong enough to activate the application of the secondary treatment, such as metal, or alternatively a rigid plastic.

At the other end of the first actuator arm will preferably be located a second actuator arm which is located substantially behind the needle shroud projection. As the needle shroud retracts, the projection causes the second actuator arm to be forced backward, in turn causing the first actuator arm to pivot about the fulcrum and actuate the application of the secondary treatment.

The second actuator arm may also preferably be able to pivot about the first actuator arm, such that as the shroud is pushed further back, the second actuator arm can pivot up and over the projection, releasing the pressure from the secondary treatment actuator and thus terminating the application of the secondary treatment.

According to a further aspect of the present invention there is provided a secondary treatment applicator substantially as described above, including

at least one reservoir capable of retaining a secondary treatment, and

a means for attaching the secondary treatment applicator to an existing treatment device.

For ease of reference throughout the specification the vaccinator shall be referred to as having only one reservoir—but again this should not be seen as limiting in any way as in other embodiments there may be two or more reservoirs as part of the present invention.

Having a greater number of reservoirs enables more than one additional treatment to be applied prior to the primary treatment.

This also enables a wider range of treatments to be delivered simultaneously, reducing the amount of time required for the treatment process.

The term “reservoir” can be taken to include a variety of forms and may be any article capable of retaining a treatment substance. For example, the apparatus may be an ink pad, liquid cartridge or spray canister and the like.

In preferred embodiments of the present invention the reservoir is an aerosol spray canister.

By the incorporation of a secondary treatment applicator including a spray-canister the present invention overcomes many of the aforementioned problems associated with marking syringes.

The spray canister is typically under pressure by a propellant, ensuring the secondary treatment is readily able to be applied once actuated.

Further, once empty or if clogged in some way, the spray canister can easily be exchanged for a new canister, reducing downtime and labour intensive maintenance of the vaccinator.

As the spray canister itself does not have any moving parts, the aforementioned problems with ink syringe systems jamming and/or not returning fully to the initial position are eliminated.

As such, large numbers of animals can be treated within a narrow timeframe with a greater ease of use and with a much reduced risk of the vaccinator jamming or applying less than the required treatment dose.

In a preferred embodiment of the present invention the spray-canister may be mounted over the top of the existing vaccinator body. However in other embodiments the reservoir may be mounted onto the side of the vaccinator or even below it.

Having the secondary treatment applicator attached to the vaccinator enables the operator greater freedom of movement (and hence safety) during animal treatment as they are not hindered by a large, often bulky reservoir earned on their back. They are also free of the associated tubes connecting the reservoir to the vaccinator.

However, it is anticipated a reservoir may be able to be mounted onto the back of the operator when a large quantity of a secondary treatment is required and where freedom of movement is not restricted, enabling a greater number of animals to be treated at any one time.

A detachable applicator also enables the user to quickly change the applicator if it becomes blocked or jams in any way.

This is a significant improvement over existing marking systems incorporated into treatment devices which often require time-consuming maintenance to repair or to clear any blockages from.

In other embodiments the secondary treatment applicator may instead be incorporated into new vaccinators as a single unit wherein the applicator may not be separable from the body of the vaccinator.

Combining a primary and a secondary treatment into the same treatment device enables an operator to treat animals one-handed, allowing the free hand to control the animal.

Applying the secondary treatment prior to applying the primary animal treatment also has a number of advantages over existing treatment devices.

The secondary treatment can be applied while the animals are still relatively calm, instead of after a sometimes painful primary treatment. This will also increase the likelihood of ensuring that all animals are correctly identified as having been treated

Applying a secondary treatment prior to applying the primary treatment has a further advantage in that the secondary treatment can have further properties rather than being solely a visible indicator, including anti-microbial activity, cleansing properties, and/or anaesthetic properties.

Having a secondary treatment with anti-microbial and/or cleaning properties allows a rapid method for preparing the animal for a treatment (such as vaccination) whilst reducing the chances of cross-infection arising from treatment of a number of animals with the same treatment device.

These properties may be a feature of the secondary treatment itself, for example the use of iodine as a visual marker, or may be a result of the mixing of the secondary treatment with an anti-microbial and/or cleansing agent.

Alternatively, the anti-microbial and/or cleansing agents may be held in a separate reservoir and delivered prior to, or simultaneously with a visual marker.

In further preferred embodiments of the present invention there may also be provided a local anaesthetic agent which may be combined with, or separate from, the secondary treatment.

Applying an anaesthetic agent prior to a primary animal treatment would result in a less painful treatment for the animal, making the animal easier to handle and ensuring optimal treatment application, in addition to reducing the risk of injury to the operator caused by a distressed animal.

It should be appreciated that although the reference throughout this specification has been made to the present invention as a marking system for use during animal treatments, the treatment apparatus and control mechanisms may be used as an applicator system in other areas.

### BRIEF DESCRIPTION OF DRAWINGS

Further aspects of the present invention will become apparent from the following description which is given by way of example only and with reference to the accompanying drawings in which:

FIG. 1 is a diagrammatic perspective view of an animal treatment and marking device in accordance with one aspect of the present invention, and

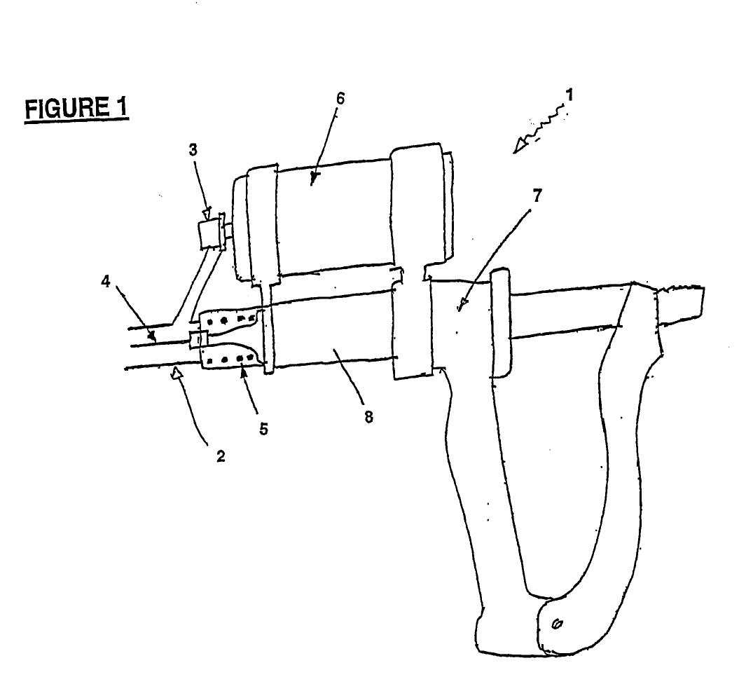

FIG. 2 is a diagrammatic perspective view of a metered dose actuator in accordance with one aspect of the present invention.

FIG. 3 is a diagrammatic representation of the mode of action of another preferred embodiment of the present invention.

### BEST MODES FOR CARRYING OUT THE INVENTION

With respect to the drawings, there is provided a marking system for use in animal treatment as indicated by arrow 1.

The marking device (1) can be separately attached to an existing vaccinator (7).

The marking system (1) incorporates a needle shroud (2), which protrudes beyond the tip of the needle (4). The shroud (2) moves longitudinally with respect to the needle (4).

This allows the needle shroud (2) to retract as the needle (4) is inserted into the animal. As the shroud (2) retracts it actuates the secondary treatment applicator (6) by movement of the nozzle attachment (3).

The marking device (1) has an aerosol spray canister (6) containing the secondary treatment. It is preferably attached to the top of the vaccinator barrel (8).

The shroud (2) is connected to an end cap device (5), which screws onto the end of the barrel (8) of the vaccinator (7).

The end cap device (5) also contains a spring (11) which forces the shroud (2) back over the needle (4) after it is withdrawn from the animal.

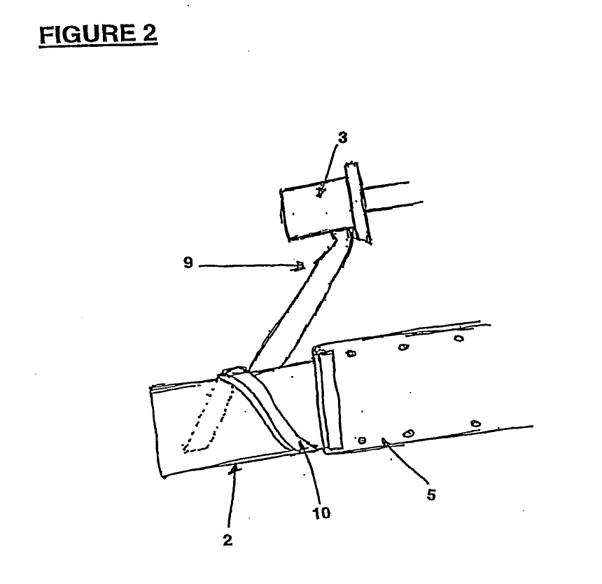

FIG. 2 shows a larger view of the needle shroud (2) and metered dose actuator (9). In preferred embodiments the shroud (2) is moulded with a helical external thread (10).

The helical thread (10) allows the shroud (2) when depressed to rotate and push the actuator (9) to apply the secondary treatment.

The thread length enables the nozzle to be actuated for a determined duration to expel a metered dose of the secondary treatment, then finish to allow the nozzle to return to its original position.

Upon removing the needle (4) from the animal the shroud (2) will rotate and return to its original position, relocating the actuator (9) onto the thread in its original location.

Holding the combined treatment/marking device in one hand the operator presses the needle shroud (2) against the site of injection on the animal. The shroud (2) begins to retract into the end cap device (5) of the vaccinator as pressure is applied.

As the shroud (2) retracts the external thread (10) of the shroud (2) moves the actuator arm (9) which activates the nozzle attachment (3) of the marking device (1), applying the secondary treatment from the aerosol canister (6) onto the site of injection.

Preferably the secondary treatment will also have sterilising and/or anaesthetic properties in addition to acting as a visible identification, sterilising the site of injection and also the needle (4) of the vaccinator (7).

The thread length enables the nozzle to be actuated for a determined duration to expel a metered dose of the secondary treatment, then finish to allow the nozzle to return to its original position.

As the operator applies further pressure the needle (4) pierces the skin and the animal is vaccinated using standard procedures.

Upon removing the needle (4) from the animal the shroud (2) will be forced back over the needle (4) by the spring in the end cap device (5). As the shroud (2) is pushed out, it rotates and returns to its original position, relocating the actuator (9) onto the thread (10) ready for the next vaccination.

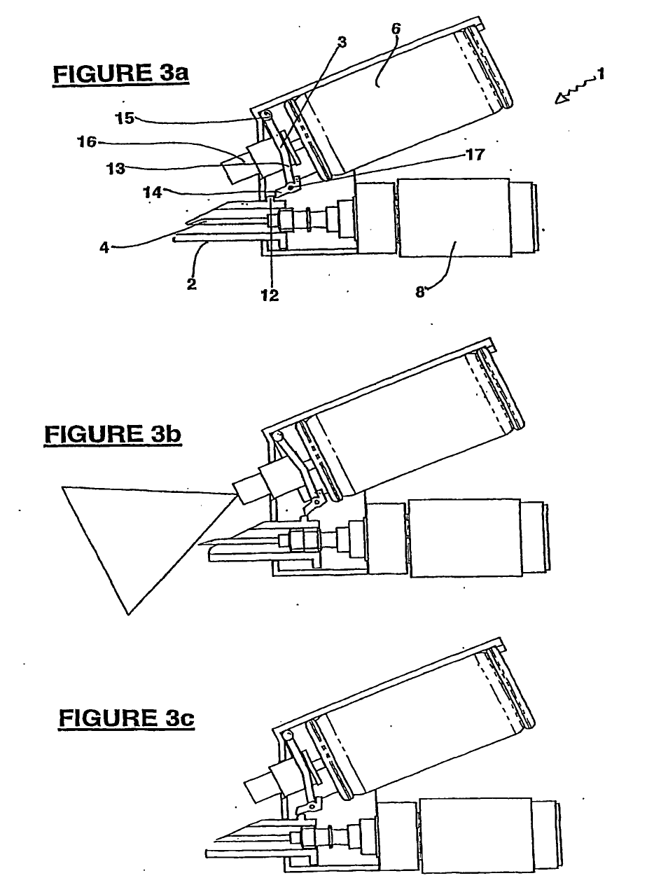

FIG. 3 shows the mechanism of action of another preferred embodiment of the present invention. In this embodiment, the needle shroud (2) has a projection (12) protruding from the body of the shroud (2).

The actuator mechanism (3) as shown by FIG. 3A consists of a first actuator arm (13) and a fulcrum (15) at one end about which the bar (13) can rotate. At the other end of the first actuator arm (13) is located a second actuator arm (14) which before use is located substantially behind the needle shroud projection (12).

The mechanism of action is shown by FIGS. 3B and 3C. As the needle shroud (2) retracts, the projection (12) causes the second actuator arm (14) to be forced backward, in turn forcing the first actuator arm (13) to pivot about the fulcrum (15) and cause the nozzle (16) to be depressed, allowing the secondary treatment to be applied from the reservoir (6).

The second actuator arm (14) will also preferably be able to pivot (17) about the first actuator arm (13), such that as the shroud (2) is pushed further back, the second actuator arm (14) pivots up and over the projection (12), causing the nozzle (16) to be released and the flow of marker from the reservoir (6) to be terminated.

Aspects of the present invention have been described by way of example only and it should be appreciated that modifications and additions may be made thereto without departing from the scope thereof as defined in the appended claims.

What we claim is:

1. A method of treating an animal with a singe animal treatment device, including
a primary treatment, and
a secondary treatment
characterised by the step of
applying the secondary treatment with the device prior to applying the primary treatment with the same device.

2. A method as claimed in claim 1 wherein the secondary treatment is applied topically.

3. A method as claimed in claim 1 or claim 2 wherein the primary treatment is applied by vaccination.

4. A method as claimed in any of claims 1 to 3 wherein the secondary treatment provides a visible identification to mark an animal receiving the primary treatment.

5. A method as claimed in any one of claims 1 to 4 wherein the secondary treatment has anti-microbial activity.

6. A method as claimed in any one of claims 1 to 4 wherein the secondary treatment has cleansing properties.

7. A method as claimed in any one of claims 1 to 4 wherein the secondary treatment has anaesthetic properties.

8. A method as claimed in any one of claims 1 to 7 for applying the secondary treatment,
characterised by the further steps of:
a) pressing the animal treatment device against an animal,
b) retracting a needle shroud,
wherein the action of retracting the shroud activates an actuator mechanism which in turn depresses a reservoir nozzle for a determined length of time to apply a quantity of the secondary treatment.

9. An animal treatment device for applying a primary and a secondary treatment including,
an actuator,
a primary treatment applicator,
a secondary treatment applicator,
characterised in that
the treatment device is configured such that as the primary treatment is initiated, the actuator mechanism activates the application of the secondary treatment prior to the application of the primary treatment.

10. An animal treatment device as claimed in claim 9 wherein the secondary treatment is applied topically.

11. An animal treatment device as claimed in either claim 9 or claim 10 wherein the primary treatment applicator is a vaccinating syringe.

12. An animal treatment device as claimed in claim 11 wherein the actuator includes a needle shroud that moves longitudinally with respect to and which protrudes beyond the needle of the vaccinating syringe.

13. An animal treatment device as claimed in claim 12 wherein the needle shroud is connected to a cap that attaches to the end of the vaccinating syringe barrel.

14. An animal treatment device as claimed in claim 12 or claim 13 wherein the actuator includes a biasing means to push the needle shroud back over the needle after treatment.

15. An animal treatment device as claimed in any one of claims 12 to 14 wherein the needle shroud is moulded with an external thread.

16. An animal treatment device as claimed in any one of claims 12 to 15 wherein the needle shroud has a projection which protrudes from the shroud at a substantially perpendicular angle.

17. An animal treatment device as claimed in any one of claims 12 to 16 wherein the actuator includes a lever that activates the application of the secondary treatment.

18. An animal treatment device as claimed in any one of claims 9 to 17 wherein the secondary treatment provides a visible identification to mark an animal receiving the primary treatment.

19. An animal treatment device as claimed in any one of claims 9 to 18 wherein the secondary treatment has anti-microbial activity.

20. An animal treatment device as claimed in any one of claims 9 to 19 wherein the secondary treatment has cleansing properties.

21. An animal treatment device as claimed in any one of claims 9 to 20 wherein the secondary treatment has anaesthetic properties.

22. An animal treatment device as claimed in any one of claims 9 to 21 wherein the secondary treatment applicator includes at least one reservoir capable of retaining a secondary treatment.

23. An animal treatment device as claimed in claim 22 wherein the reservoir is an aerosol spray canister.

24. An animal treatment device as claimed in claim 22 or claim 23 wherein the reservoir may be mounted over the top of an existing vaccinating syringe.

25. An animal treatment device as claimed in any one of claims 22 to 24 wherein the secondary treatment applicator is a separate applicator, able to be attached to an existing vaccinating syringe.

26. An animal treatment device as claimed in claim 25 wherein the secondary treatment applicator is detachable from the device.

27. A method substantially as described herein with reference to and as illustrated by the accompanying description and drawings.

28. An animal treatment device substantially as described herein with reference to and as illustrated by the accompanying description and drawings.
